(19) United States
(12) Patent Application Publication
(10) Pub. No.: US 2020/0400664 A1
(43) Pub. Date: Dec. 24, 2020

(54) NiO CHIP AND THE PREPARING METHOD AND USE THEREOF

(71) Applicant: National Chiao Tung University, Hsinchu (TW)

(72) Inventors: Bor-Ran Li, Hsinchu (TW); Yu-Ling Hsieh, Taipei (TW); Chien-Wei Chen, New Taipei (TW); Wan-Hsuan Lin, Taipei (TW)

(21) Appl. No.: 16/836,913

(22) Filed: Mar. 31, 2020

(30) Foreign Application Priority Data
Jun. 21, 2019 (TW) 108121634

(51) Int. Cl. G01N 33/553; G01N 33/58

(52) U.S. Cl. CPC G01N 33/553; G01N 33/582

(57) ABSTRACT

An embodiment of this invention A NiO chip is provided. The NiO chip includes a substrate, a nickel oxide thin film on the substrate, and a bioprobe layer on the nickel oxide thin film. The nickel oxide thin film has a light transmittance of more than 60% and a nanostructure. The bioprobe layer includes a plurality of bioprobes modified by Histidine (His) or a His-tagged protein.

### CROSS-REFERENCE TO RELATED APPLICATION

This application claims priority from Taiwan Application No. 108121634, filed on Jun. 21, 2019, in the Taiwan Intellectual Property Office, the content of which is hereby incorporated by reference in their entirety for all purposes.

### BACKGROUND

### 1. Technical Field

The invention relates to a nickel oxide chip, a preparation method and application thereof, and particularly to a nickel oxide chip having a bioprobe disposed thereon, a preparation method and application thereof.

### 2. Description of the Related Art

Most bioprobes have specific binding sites with an analyte. Therefore, how to modify the surface of orientated bioprobes and retain its activity at the same time is one of the important key issues affecting the detection sensitivity when developing a biochip or a biodetector. However, it is still a challenge to effectively modify the bioprobes and take these conditions into consideration.

In recent years, among many methods for fixing orientated biomolecules to the surfaces of biochips, the self-assembled monolayer technology has been considered as a potential modification method. The self-assembled monolayer technology is mainly immersing a substrate in a solution in which surface-active organic and/or inorganic molecules are dissolved, so that the organic and/or inorganic molecules in the solution can spontaneously form a bond with the substrate to chemically adsorb on the substrate surface to form a single layer. Further, when the surface of the substrate is a metal surface, the molecules can be tightly bound to the substrate through metal chelation, and the assembled molecules have controllability and orientation, so they can be widely used in various fields.

### SUMMARY

In view of the foregoing, an aspect of the invention is to provide a high-efficiency protein modification rate, high sensitivity, simple operation, and low cost, which can perform high-throughput detection and analysis with only a few bioprobes and samples, and can be applied to nickel oxide chips for a variety of biological target detection and/or optical detection.

According to an aspect of the invention, a nickel oxide chip is provided, comprising: a substrate; a nickel oxide thin film formed on the substrate and having a light transmittance of more than 60% and a nanostructure; and a bioprobe layer disposed on the nickel oxide thin film, wherein the bioprobe layer comprises a plurality of bioprobes modified by histidine (His) or a His-tagged protein.

In an embodiment of this invention, the nickel oxide thin film has a thickness of 50 nm to 150 nm.

In an embodiment of this invention, the histidine or the His-tagged protein is disposed on the nickel oxide thin film by self-assembled monolayer technology.

According to another aspect of the invention, a nickel oxide chip is provided. The nickel oxide chip comprises: a substrate; a nickel oxide thin film formed on the substrate; and a bioprobe layer disposed on the nickel oxide thin film. The nickel oxide thin film is formed by the following steps: forming a nickel thin film on the substrate; and calcinating the nickel thin film at a temperature more than 500° C. for a predetermined time to form the nickel oxide thin film. The bioprobe layer comprises a plurality of bioprobes modified by histidine (His) or a His-tagged protein.

In an embodiment of this invention, the nickel thin film has a thickness of 1 nm to 1000 nm.

In an embodiment of this invention, the predetermined time is less than 1 hour.

According to another aspect of the invention, a method of preparing a nickel oxide chip is provided. The method comprises: forming a nickel thin film on the substrate; calcinating the nickel thin film at an annealing temperature to form a nickel oxide thin film; and disposing a plurality of bioprobes on the nickel oxide thin film via self-assembled monolayer technology by a histidine or a His-tagged protein.

In an embodiment of this invention, the annealing temperature is more than 500° C.

In an embodiment of this invention, the nickel thin film is calcinated for less than 1 hour.

According to another aspect of the invention, a nickel oxide chip used to the application of bio-detection and/or optical detection is provided.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The invention will become more apparent to those with ordinary skills in the art with reference to the following detailed description in conjunction with the accompanying drawings, among which:

FIG. 1 is a diagram of a nickel oxide chip according to an embodiment of the invention.

FIG. 2 is a diagram of the structure of a bioprobe layer of a nickel oxide chip according to an embodiment of the invention.

FIG. 3 is a diagram of a flowchart of a method for preparing a nickel oxide chip according to an embodiment of the invention.

FIG. 4 is a diagram of the SEM images of the nickel oxide thin film in a nickel oxide chip according to an embodiment of the invention and the nickel oxide thin film of a comparative example.

FIG. 5 shows the transmittance of the nickel oxide thin film in the nickel oxide chip according to some embodiments of the invention and the nickel oxide thin film of the comparative example.

FIG. 0.6 shows the protein loading ratio of the nickel oxide thin film in the nickel oxide chip according to the embodiment of the invention and the nickel oxide thin film of the comparative example.

FIG. 7 is a comparison diagram of the nickel oxide thin films prepared by different heat treatment times.

FIG. 8 is a fluorescence intensity diagram of a Histidine-labeled green fluorescent protein on a nickel oxide chip in a solution of different pH value according to an embodiment of the invention.

FIG. 9 is a diagram of the nickel oxide chip examined with a conjugate focus microscope according to some embodiments of the invention.

Part (a) of FIG. 10 is a diagram showing the results of detecting the HER2 protein concentration using a nickel oxide chip according to an embodiment of the invention, and part (b) of FIG. 10 is a diagram showing the results of detecting the HER2 protein concentration by an enzyme immunoassay.

FIG. 11 is a diagram of a result of detecting specificity of HER2 protein by using the nickel oxide chip according to an embodiment of the invention.

### DETAILED DESCRIPTION

The invention aims to develop a nickel oxide chip for biological detection with a simple manufacturing process and low cost, but with a sensitive signal and large-scale production potential. A preparation method and application of the nickel oxide chip are also provided.

FIG. 1 is a diagram depicting a nickel oxide chip according to an embodiment of the invention. Referring to FIG. 1, according to an embodiment of the invention, a nickel oxide chip 10 includes a substrate 100, a nickel oxide thin film 103, and a bioprobe layer 105. In one embodiment, the nickel oxide thin film 103 may have a light transmittance and a nano structure. In another embodiment, the nickel oxide thin film 103 may be a nickel oxide thin film formed by annealing a nickel thin film at a predetermined temperature for a predetermined time. In one embodiment, the nickel thin film may have a thickness of 1 nm to 1000 nm, or a thickness of 1 nm to 100 nm. The nickel oxide thin film 103 may have a thickness of 50 nm to 150 nm.

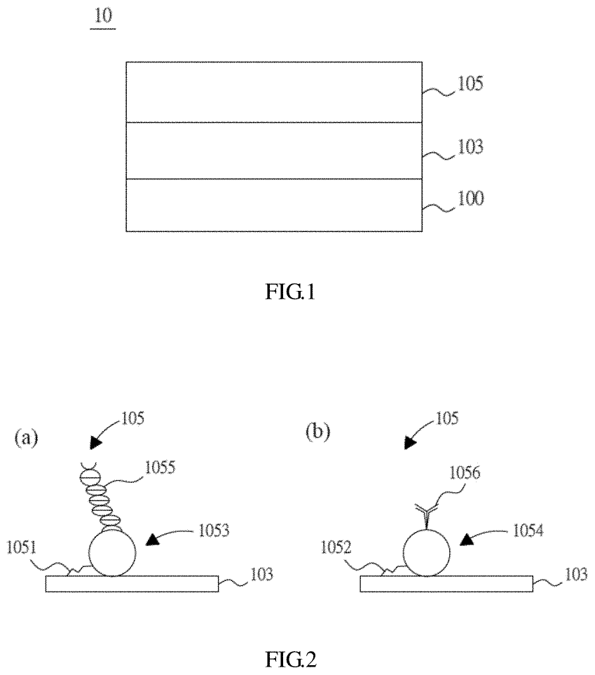

The specific structure is shown in FIG. 2. The bioprobe layer 105 includes a plurality of bioprobes disposed on the nickel oxide thin film through a Histidine or a Histidine-labeled protein using a self-assembled disposed single-layer membrane technology. FIG. 2 is a diagram of the structure of a bioprobe layer 105 of a nickel oxide chip 10 according to an embodiment of the invention. Referring to part (a) of FIG. 2, the bioprobe layer 105 includes a plurality of DNA probes 1055 disposed on the nickel oxide thin film 103 through a Histidine-labeled protein 1053 with Histidine 1051. In this example, the histidine-labeled protein 1053 can be a single streptavidin. By utilizing the high affinity of monomeric streptavidin and biotin, the DNA probe 1055 can be orientationally fixed onto the surface of the nickel oxide thin film 103 and will have the correct orientation. Fixing the bioprobe to the surface of the chip can further reduce the non-specific binding, which can greatly increase the number of effective DNA probes 1055 on the surface of the nickel oxide thin film 103. Further referring to part (b) of FIG. 2, the bioprobe layer 105 includes a plurality of antibodies 1056 disposed on the nickel oxide thin film 103 through a Histidine-labeled protein 1054 with Histidine 1052. In this example, the histidine-labeled protein 1054 can be a G protein. Through the high affinity between the G protein and the Fc end of the antibody, the required antibody 1056 can be orientationally fixed to the surface of the nickel oxide thin film. The correctly orientated bioprobe can be fixed onto the surface of the chip to improve the traditional physical adsorption having the drawbacks arrised from the non-specific adsorption. Thus, the number of effective antibodies 1056 adsorbed on the surface of the nickel oxide thin film is greatly increased.

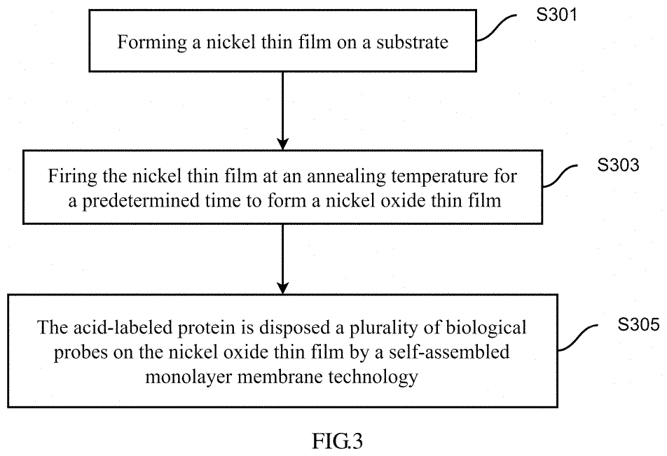

FIG. 3 is a diagram of a flowchart of a method for preparing a nickel oxide chip according to an embodiment of the invention. Referring to FIG. 1, a method for preparing a nickel oxide chip according to the embodiment of the invention includes the following steps. In step S301, a nickel thin film is formed on a substrate. In step S303, the nickel thin film is calcinated at an annealing temperature for a predetermined time to form a nickel oxide thin film. In step S305, a plurality of bioprobes are disposed on the nickel oxide thin film through histidine or histidine-labeled protein by a self-assembled monolayer membrane technology.

In step S301, any suitable method may be used to form a nickel thin film on the substrate. Examples of the substrate may include, but are not limited to, a glass substrate, a quartz substrate, and a silicon substrate. The thickness of the substrate is not particularly limited. In an embodiment, before the step S301, a step of cleaning the substrate may be further included to remove impurities on the surface of the substrate. The nickel thin film is formed on the substrate after the impurities on the surface of the substrate are removed. A method suitable for forming the nickel thin film includes, but is not limited to, thermal evaporation, sputtering, pulse laser deposition, chemical vapor deposition (CVD), plasma assist chemical vapor deposition, screen printing, electroplating, spray cracking, spin coating, liquid deposition, etc. In an embodiment, a chemical vapor deposition is used to form a nickel thin film on the substrate. The nickel thin film may have a thickness of 1 nm to 1000 nm.

After step S301 is completed, the formed nickel thin film is cleaned and then calcinated at an annealing temperature for a predetermined time to form a nickel oxide thin film (step S303). The annealing temperature may be in a range of about 500° C. or higher, about 600° C. or higher, about 700° C. or higher, about 800° C. or higher, about 900° C. or higher, about 1000° C. or higher, or about 1100° C. or higher. In some embodiments, the annealing temperature is above 800° C. In some other embodiments, the annealing temperature is above 1100° C. When the nickel thin film is calcinated at an annealing temperature of 500° C. or higher, the nickel thin film will become a nickel oxide thin film having a light transmittance of 60% or more. When the nickel thin film is further calcinated at an annealing temperature of 800° C. or higher, the nickel thin film will further form nickel oxide with a nano structure to increase the surface area/volume ratio, and the surface area for fixing histidine protein is thus increased to achieving the goal of increasing the final modification amount of the bioprobe. When the annealing temperature is 1100° C. or higher, the transmittance of the nickel oxide thin film can be further improved. According to some embodiments of the invention, the calcining time for preparing a nickel oxide chip may be generally less than about 1 hour. For example, the calcinating time may be in a range of about 30 to 150 seconds, may be in a range of about 60 to 150 seconds, may be in a range of about 60 to 120 seconds, or may be about 120 seconds. Compared with the nickel thin film, the calcined nickel oxide thin film can have a larger thickness. In some embodiments, when the thickness of the nickel thin film is about 50 nm, the calcined nickel oxide thin film may have a thickness of about 150 nm.

Next, step S305 is performed. Using the high affinity between the nickel oxide and histidine, a plurality of bioprobes are disposed on the nickel oxide thin film by self-assembled monolayer membrane technology through histidine or histidine-labeled protein. In this step, the histidine-labeled protein is not specifically limited, and the histidine-labeled protein may be any proteins labeled with histidine. Examples of histidine-tagged proteins include, but are not limited thereto, histidine-tagged single-streptavidin, histidine-tagged G proteins, or histidine-tagged antibodies. The bioprobe used may optionally select a DNA probe, an antibody, or a combination thereof. Specific examples are provided below to further illustrate that the nickel oxide thin film of the nickel oxide chip of the invention has the advantages of increased protein loading rate, light transmittance, and the like, so that the sensitivity and other characteristics of the nickel oxide chip can be further improved.

### Preparation of Nickel Oxide Thin Film

### Example 1

1. The glass substrate is rinsed with 75% alcohol, blow dry with a nitrogen gun, and then is put into an oxygen plasma machine to remove impurities on the surface of the glass substrate. The parameters of the oxygen plasma were set to 1 mbar (0.5 liter/hour), 60 W, and 120 seconds.

2. A 50 nm nickel thin film is deposited on the surface of the cleaned glass substrate by CVD. The glass substrate coated with a nickel film is cleaned with deionized water and 75% alcohol, and then the surface moisture is dried with a nitrogen gun.

3. The nickel thin film was calcined at an annealing temperature of 800° C. for 120 seconds to obtain a nickel oxide thin film 1.

### Example 2

A nickel oxide thin film 2 was prepared in the same manner as in Example 1 except that the nickel thin film was calcined at an annealing temperature of 1100° C.

### Comparative Example 1

A nickel oxide thin film was prepared in the same manner as in Example 1 except that the nickel thin film was calcined at an annealing temperature of 300° C.

### Comparative Example 2

A nickel oxide thin film was prepared in the same manner as in Example 1 except that the nickel thin film was calcined at an annealing temperature of 400° C.

### Comparative Example 3

A 0.08 mm nickel foil was directly taken, and a nickel thin film was calcined at an annealing temperature of 800° C. for 1 hour to obtain a comparative nickel oxide thin film.

Surface Morphology of Nickel Oxide Thin Film

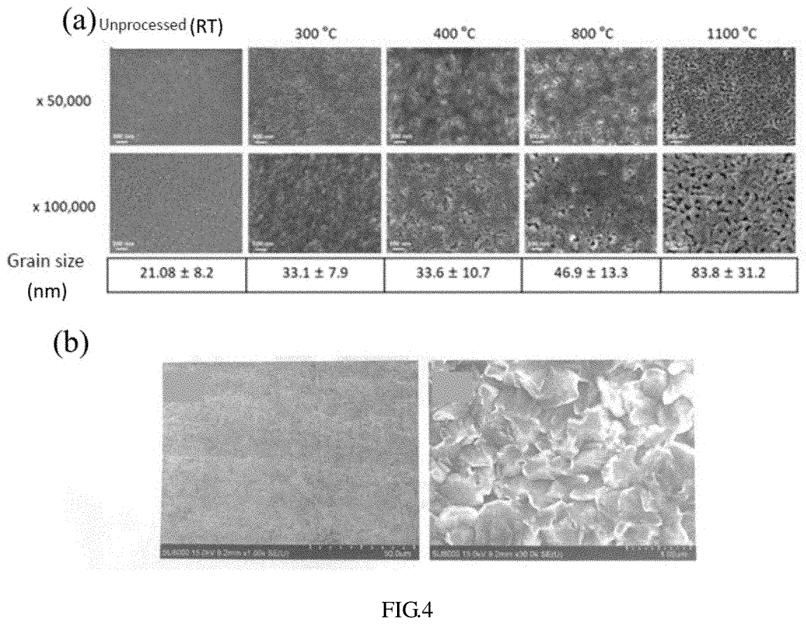

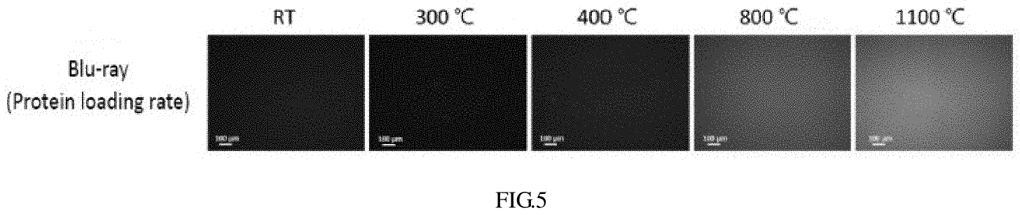

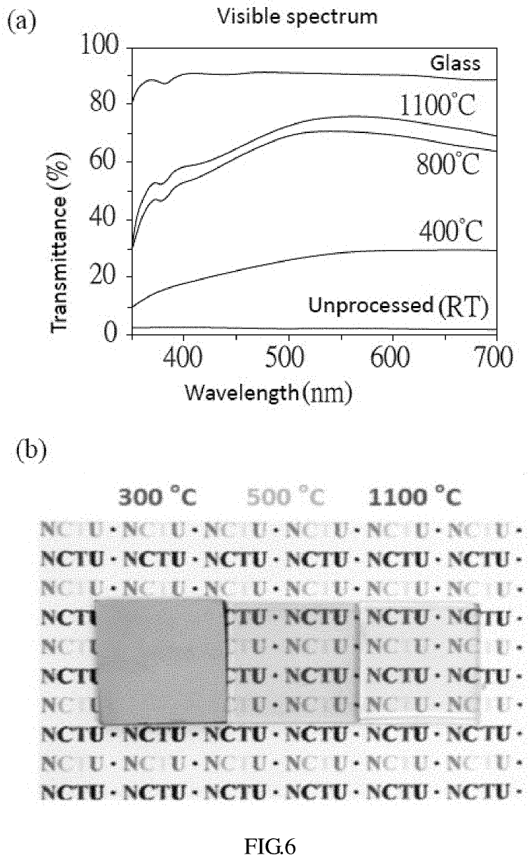

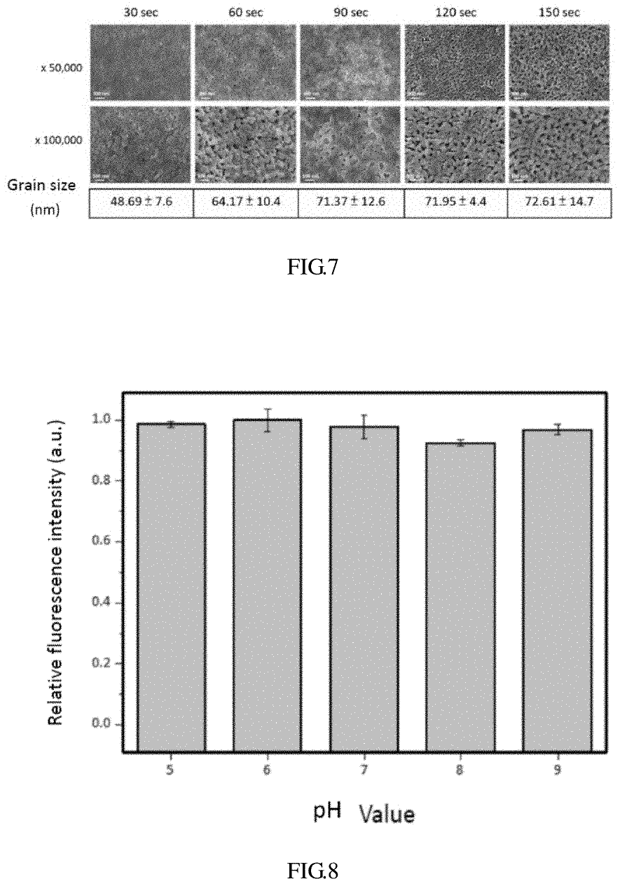

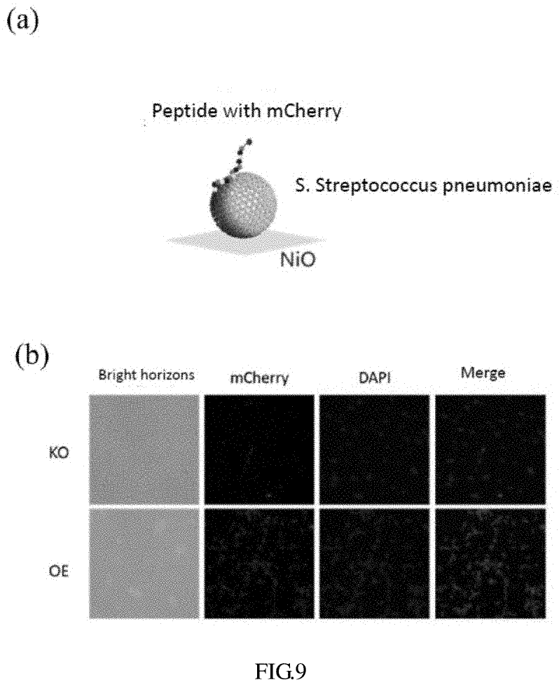

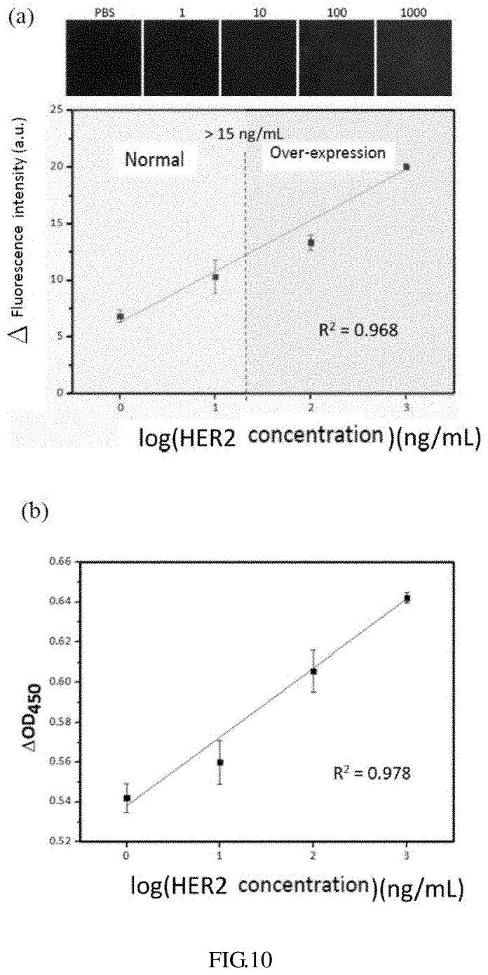

The surface morphology of the nickel oxide thin film was observed with a scanning electron microscope (SEM), and the results obtained are shown in part (a) of FIG. 4, which shows SEM images of the nickel oxide thin film of Examples 1 and 2 and Comparative Examples 1 and 2. Part (b) of FIG. 4 shows SEM images of the nickel oxide thin film of Comparative Example 3. It can be seen from FIG. 4 that the nickel oxide thin films formed in Examples 1 and 2 and Comparative Examples 1 to 3 have different morphologies, and only the nickel oxide thin films of Examples 1 and 2 have a nanostructure. Accordingly, the nickel oxide thin film of the embodiments of this invention can improve the modification rate of the bioprobe on the surface of the nickel oxide thin film by the nanostructure to enhance the final detection signal.

Evaluation of Protein Loading Rate of Nickel Oxide Thin Film

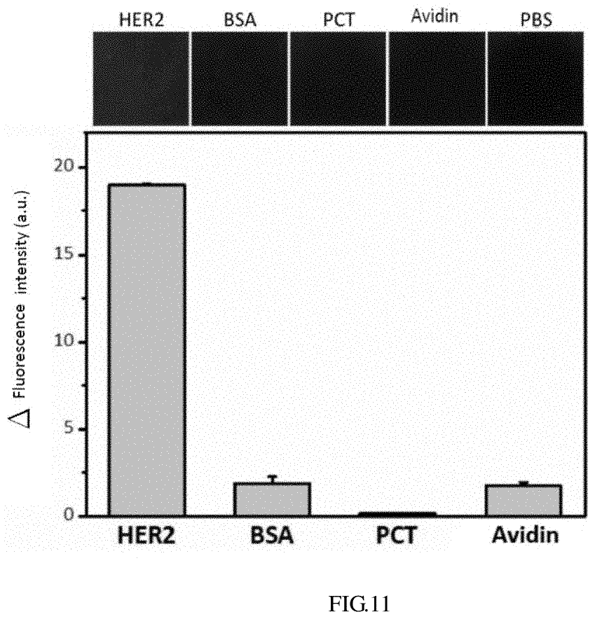

Using the nickel thin film as a control, the protein loading rates of the nickel oxide thin films of Examples 1 and 2 and Comparative Examples 1 and 2 were evaluated. FIG. 5 shows the transmittance of the nickel oxide thin film in the nickel oxide chip according to some embodiments of the invention and the nickel oxide thin film of the comparative example. As can be seen from FIG. 5, compared to the comparative example, the nickel oxide thin films of Examples 1 and 2 have higher protein loading rates, and the nickel oxide chip produced at an annealing temperature of 1100° C. has a higher protein loading rate then the nickel oxide chip produced at an annealing temperature of 800° C. That is, compared with the comparative example, the nickel oxide thin film of the invention can increase the number of bioprobes per unit area, thereby improving the detection sensitivity of the nickel oxide chip of the invention.

Evaluation of Transmittance of Nickel Oxide Thin Film

A glass was used as a control to evaluate the transmittance of the nickel oxide thin films of Examples 1 and 2 and Comparative Examples 1 and 2. The results obtained are shown in FIG. 6. FIG. 6 shows the protein loading ratio of the nickel oxide thin film in the nickel oxide chip according to the embodiment of the invention and the nickel oxide thin film of the comparative example. In part (a) of FIG. 6, compared with the comparative example, the nickel oxide thin films of Examples 1 and 2 have a light transmittance of more than 60%. In part (b) of FIG, the light transmittance of the nickel oxide chip calcined at 1100° C. is greater than that of the nickel oxide thin film calcined at an annealing temperature of 800° C.

From the above evaluation of characteristics, it can be seen that the effect is better when the annealing temperature is greater than 1100° C. Next, the annealing temperature was 1100° C., and the time for the heat treatment was compared. The comparison result is shown in FIG. 7. FIG. 7 is a comparison diagram of nickel oxide thin films prepared by different heat treatment times. It can be seen from FIG. 7 that the grain size will increase as the heat treatment time increased, and the coral-like nanostructure is becoming clearer. The grown coral-like nanostructure has a better specific surface area and provides more surface area to be modified by histidine-tagged proteins, so that the final detection signal can be augmented.

The advantages of the invention will be explained further by taking a nickel oxide chip containing the nickel oxide thin film 2 as an example.

pH Stability

After the glass substrate having the nickel oxide thin film 2 thereon was immersed in a solution of pH 5-9 overnight, the surface of the glass substrate was washed with deionized water the next day, and then immersed in histidine-labeled green fluorescent protein (GFP) solution, after the histidine-labeled green fluorescent protein (His₆-GFP) was fixed on the nickel oxide thin film 2, the fluorescence intensity was measured to determine its stability. FIG. 8 is a fluorescence intensity diagram of a histidine-labeled green fluorescent protein on a nickel oxide chip in a solution of different pH value according to an embodiment of the invention. It can be seen from FIG. 8 that the nickel oxide chip has good stability in the range of pH value 5 to 9, according to an embodiment of the invention.

Feasibility of Optical Inspection Applications

In a similar manner to the above, two kinds of Streptococcus pneumoniae that overexpressed (OE) and knock out (KO) SMU290 membrane proteins were fixed onto a glass substrate having a nickel oxide thin film 2 thereon, and then a peptide with m-cherry red fluorescent protein and specific to SMU290 membrane protein is cultivated. The structure of the completed nickel oxide chip is shown in part (a) of FIG. 9. Part (a) of FIG. 9 is a diagram of a nickel oxide chip according to some embodiments of the invention. The above nickel oxide chip was examined with a conjugate focus microscope, and the results obtained are shown in part (b) of FIG. 9. Part (b) of FIG. 9 is a diagram of the results of the nickel oxide chip examined with a conjugate focus microscope according to some embodiments of the invention. From FIG. 9, it can be observed that whether the colonial morphology under white light, the DNA position of the bacteria indicated by DAPI under blue light, or the SMU290 membrane protein labeled with peptides under red light are all very clear. The results show that the feasibility of developing the Ni0 chips in other optical inspection applications.

Feasibility of Bioassay Applications

A glass substrate having a nickel oxide thin film 2 thereon was immersed in a histidine-labeled γGB1 protein (His₆-γGB1) with an antibody Herceptin thereon to form a nickel oxide chip for human epidermal growth factor receptor 2 (HER2). The results are shown in FIGS. 10 and 11. Part (a) of FIG. 10 is a diagram showing the results of detecting the HER2 protein concentration using a nickel oxide chip according to an embodiment of the invention, and part (b) of FIG. 10 is a diagram showing the results of detecting the HER2 protein concentration by an enzyme immunoassay, and FIG. 11 is a diagram of a result of detecting specificity of HER2 protein by using the nickel oxide chip according to an embodiment of the invention. It can be seen from FIG. 10 that the detection sensitivity of the nickel oxide chip covers the range of clinical needs (>15 ng/mL), and the coefficient of determination (R²) reaches 0.96, which is in line with enzyme immunity, according to the embodiment of the invention. The analytical test results match, but it has the advantage of requiring only a small amount of antibodies and samples to achieve high sensitivity detection. From FIG. 11, it can be seen that the nickel oxide chip is highly specific for detecting HER2 protein, according to the embodiment of the invention.

The above various evaluations and tests of the nickel oxide chips of the embodiments of the invention confirm that the nickel oxide chips of the embodiments of the invention can modify the surface of the nickel oxide chips in a targeted manner through the mechanism of self-assembled single-layer films, and thus improve the disadvantages of not being able to be orientated by physically adsorbing and immobilizing proteins, which may cause protein denaturation, complicated steps that require multiple chemical modifications, and chemical pretreatment of proteins. And as long as it is a histidine protein, or any protein labeled with histidine, which can be fixed to the surface of the nickel oxide chip in the above-mentioned manner. Therefore, an operator does not need professional training, and the protein can be quickly modified according to the needs of the time and place. Because the fixing method is in a form of metal chelation, it can maintain the protein configuration intact to maintain its activity without the need for any chemical pretreatment, so that the number of the effective bioprobe that is finally fixed on the surface of the chip can be improved. Furthermore, the nickel oxide chip of the embodiment of the invention has a nano structure, which can increase the protein loading rate by its physical advantages, and has a high-performance protein modification rate, which can further enhance the final detection signal to achieve the benefits that only a small number of samples are needed to perform high-throughput assays. Further, compared with the method of generating orientated bioprobe in the past, the invention has the characteristics of low cost, simple operation and manufacturing process, and the like.

The above description is exemplary only, and not restrictive. Any equivalent modification or change made without departing from the spirit and scope of the invention shall be included in the scope of the attached patent application.

What is claimed is:

1. A nickel oxide chip, comprising:
a substrate;
a nickel oxide thin film formed on the substrate and having a light transmittance of more than 60% and a nanostructure; and
a bioprobe layer disposed on the nickel oxide thin film, wherein the bioprobe layer comprises a plurality of bioprobes modified by histidine (His) or a His-tagged protein.

2. The nickel oxide chip of claim 1, wherein the nickel oxide thin film has a thickness of 50 nm to 150 nm.

3. The nickel oxide chip of claim 1, wherein the histidine or the His-tagged protein is disposed on the nickel oxide thin film by self-assembled monolayer technology.

4. The nickel oxide chip of claim 1, wherein the bioprobes are DNA probes or antibodies.

5. A nickel oxide chip, comprising:
a substrate;
a nickel oxide thin film formed on the substrate, wherein the nickel oxide thin film is formed by the following steps:
forming a nickel thin film on the substrate; and
calcinating the nickel thin film at a temperature more than 500° C. for a predetermined time to form the nickel oxide thin film; and
a bioprobe layer disposed on the nickel oxide thin film, wherein the bioprobe layer comprises a plurality of bioprobes modified by histidine (His) or a His-tagged protein.

6. The nickel oxide chip of claim 5, wherein the nickel thin film has a thickness of 1 nm to 1000 nm.

7. The nickel oxide chip of claim 5, wherein the predetermined time is less than 1 hour.

8. The nickel oxide chip of claim 5, wherein the bioprobes are DNA probes or antibodies.

9. The nickel oxide chip of claim 5, wherein the His-tagged protein is His-tagged γGB1 protein.

10. A method of preparing a nickel oxide chip, comprising:
forming a nickel thin film on the substrate;
calcinating the nickel thin film at an annealing temperature to form a nickel oxide thin film; and
disposing a plurality of bioprobes on the nickel oxide thin film via self-assembled monolayer technology by a histidine or a His-tagged protein.

11. The method of preparation of claim 10, wherein the annealing temperature is more than 500° C.

12. The method of preparation of claim 10, wherein the nickel thin film is calcinated for less than 1 hour.

13. A method of using a nickel oxide chip, comprising:
providing a nickel oxide chip having a substrate and a nickel oxide film disposed on the substrate;
fixing a bacteria on the nickel oxide film, wherein the bacteria expresses a specific membrane protein;
culturing the bacteria by a culture medium containing a peptide specifically binding the specific membrane protein, wherein the peptide is modified by a fluorophore; and
detecting fluorescence generated from the fluorophore with a conjugate focus microscope.

14. A method of using a nickel oxide chip, comprising:
providing the nickel oxide chip of claim 1, wherein the bioprobe layer comprises an antibody modified by a His-tagged protein;
immersing the nickel oxide chip in a solution of a protein being specifically recognized by the antibody; and
determining the amount of the protein in the solution.
